(19) United States
(12) Patent Application Publication
(10) Pub. No.: US 2026/0209313 A1
(43) Pub. Date: Jul. 23, 2026

(54) LAMININ EXTRACTION, PURIFICATION AND POLYMERIZATION PROCESSES, USE, POLYLAMININ AND KIT

(71) Applicant: CRISTÁLIA PRODUTOS QUÍMICOS FARMACÊUTICOS LTDA., Itapira (BR)

(72) Inventors: Ogari de Castro PACHECO, Itapira (BR); Francesco Brugnera TEIXEIRA, Itapira (BR); German Enrique WASSERMANN, Itapira (BR); Aline Hunger RIBEIRO, Itapira (BR)

(21) Appl. No.: 19/141,997

(22) PCT Filed: Dec. 19, 2023
(86) PCT No.: PCT/BR2023/050461

§ 371 Date: Jun. 20, 2025

(30) Foreign Application Priority Data
Dec. 21, 2022 (BR) 1020220262764

(51) Int. Cl. C07K 14/78; A61K 38/00; B01D 15/34; B01D 15/36; B01D 15/42; C07K 1/18; C07K 1/30; C07K 1/34

(52) U.S. Cl. CPC C07K 14/78; B01D 15/34; B01D 15/362; B01D 15/424; C07K 1/18; C07K 1/303; C07K 1/34; A61K 38/00

(57) ABSTRACT

Process for extraction and purification of laminins from human placenta, with preserved acid polymerization capacity to generate polylaminin, are described. The process of acid polymerization of purified laminins, the use of purified laminins in the manufacture of polylaminin pharmaceutical compositions, as well as a kit for extemporaneous preparation of said compositions, wherein the compositions and kit are suitable for the therapy of central nervous system injuries, mainly spinal cord injuries, are also described.

### FIELD OF INVENTION

The present invention is in the field of protein extraction and purification processes for therapeutic purposes, more precisely it refers to laminins extraction and purification from human placenta, with preserved acid polymerization capacity to generate polylaminin. The invention also relates to the acid polymerization process of purified laminins, the use of purified laminins in the manufacture of polylaminin pharmaceutical compositions as well as a kit for the extemporaneous preparation of said compositions, in which the compositions and the kit are suitable for the therapy of central nervous system injuries, mainly spinal cord injuries.

### BACKGROUND OF THE INVENTION

According to the Brazilian Ministry of Health (MoH), “spinal cord injury is one of the most serious and dramatic injuries that can affect humans, with enormous physical, psychological, and social consequences. We name spinal cord injury any damage to the structures contained within the spinal canal (medulla, conus medullaris, and cauda equina), which can lead to motor, sensory, autonomic, and psycho-affective alterations”. In 2013 and 2015, MoH stated that the annual global incidence of spinal trauma is about 15 to 40 cases per million inhabitants, and, in Brazil, it has been estimated that more than 10,000 new cases of spinal cord injury occur each year, being trauma the main cause (Brazil. Ministry of Health. Health Care Secretariat. Programmatic Strategic Actions Department. Guidelines on Caring for People with Spinal Cord Injury/Ministry of Health, Health Care Secretariat, Programmatic Strategic Actions Department and Specialized Care Department.—2^{nd}. ed—Brasilia: Ministry of Health, 2015. 68 p.: il. ISBN 978-85-334-2229-2; Brazil. Ministry of Health. Health Care Secretariat. Programmatic Strategic Actions Department. Guidelines on Caring for People with Spinal Cord Injury/Ministry of Health, Health Care Secretariat, Programmatic Strategic Actions Department and Specialized Care Department.—Brasilia: Ministry of Health, 2013. 68 p.: il. ISBN 978-85-334-2025-0). Worldwide, the World Health Organization (WHO) estimates that between 250,000 and 500,000 people suffer a spinal cord injury each year. In addition to the obvious physical and psychosocial damages to the individual, which includes a two to five times more likely to die prematurely, especially in low- and middle-income countries, this type of injury also results in substantial individual and social costs (https://www.who.int/news-room/fact-sheets/detail/spinal-cord-injury, accessed in Nov. 7, 2022).

MoH also recommends that any patient with polytrauma needs to be treated with special care since the first aid at the scene of the accident, when he/she must be adequately immobilized, as he/she is considered suspected of having a spinal cord injury. Regarding to the drug treatment, methylpredinisolone administration is not currently recommended for patients with spinal trauma, despite its use has been reported in some studies. After initial care, such as the use of a cranial halo in cervical fractures/dislocations and rest in thoracolumbar fractures, definitive surgical treatment of fractures is the recommendation for clinically stable patients as soon as possible.

Given this scenario, it is clear that the treatment for central nervous system injuries, mainly spinal cord injuries, still represents a challenge, since there is no spontaneous regenerative process and, basically, there are no therapeutic strategies, as there is currently no treatment able to reverse spinal cord injuries.

To overcome this lack, multiple approaches have been identified in the state of the art in an attempt to regenerate nervous tissue that has been subjected to trauma and to promote the reconstruction of spinal cord injuries. Some of these efforts include, for example, the therapeutic application of the protein laminin.

Laminin, or laminins, are large extracellular glycoproteins from a family of structurally homologous isoforms that constitute the main component of basement membranes. Laminins have a cruciform shape composed of three different polypeptide chains (alpha-α, beta-β and gamma-γ), which are held together by alpha-helix interactions, and disulfide bonds, and have the capacity for self-organization. The different combinations of alpha, beta, and gamma chains result in 15 different known laminin isoforms, which confer specificities to the tissues that contain them.

An observed of laminins, property useful in therapeutic application, is the ability to polymerize to form a polymer, or a protein aggregate. While in vivo laminin polymerization is facilitated and related to its ability to adhere to the surface of the plasma membrane by binding to cognate integrins, α-dystroglycan, and sulfated glycolipids, it has been observed that laminin polymerizes r specific conditions in vitro. Yurchenco et al. (1985) described the strict need for a critical laminin concentration of 140 nM (0.1 mg/mL) for self-polymerization and formation of large three-dimensional networks in vitro, while, later, other authors have demonstrated the polymerization below the critical concentration and in specific pH conditions, more precisely in acidic medium (Yurchenco, P. D., Tsilibary, E. C., Charonis, A. S., & Furthmayr, H. (1985). Laminin polymerization in vitro. Evidence for a two-step assembly with domain specificity. The Journal of Biological Chemistry, 260 (12), 7636-7644). Usually, in the literature, the terms “polymerization” and “polymer” are used to refer to these structures with several associated laminin units. (Yurchenco, P. D., & Cheng, Y.-S. (1993). Self-assembly and Calcium-binding Sites in Laminin. The Journal of Biological Chemistry, 268 (23), 17286-17299).

Obtaining laminin polymers from a mixture of laminin in acidic medium, regardless of critical concentration, was described in the state of the art by Freire and Coelho-Sampaio, in 2000 (Freire, E., & Coelho-Sampaio, T. (2000). Self-assembly of Laminin Induced by Acidic pH. Journal of Biological Chemistry, 275 (2), 817-822). In this article, the authors examined the relationship between lipid surface charge and laminin polymerization. During the investigations, they observed that the acidic pH induces the aggregation of laminin in solution in the presence of divalent cations, in an organized process and not a simple non-specific precipitation of the protein at its isoelectric point, even in the absence of a solid substrate for adsorption. In 2002, Freire associated the structural properties of laminin matrices specifically polymerized in acidic medium with neuritogenesis (Freire, E. (2002). Structure of laminin substrate modulates cellular signaling for neuritogenesis. Journal of Cell Science, 115 (24), 4867-4876). According to the document, these matrices would be morphologically similar to those found in vivo on surfaces permissive to axonal growth, favoring neuritogenesis over cell proliferation, and pointed out to the possibility of using acidic laminin matrices to stimulate axonal regeneration.

However, the above-mentioned documents failed to describe pharmaceutical compositions comprising laminins and the therapeutic use of laminin polymers in nervous tissue injuries, mainly in spinal cord injuries. In this regard, the Brazilian patent application PI 0805852-0, entitled “Protein acid polymers, production processes, use of protein acid polymers, pharmaceutical composition and treatment method” refers to the application of laminin polymers in pharmaceutical compositions with regenerative and anti-inflammatory activity, especially for the treatment of animals affected by traumatic, degenerative, or inflammatory tissue injuries in the nervous, muscular, epithelial and connective tissues. The document includes, regarding compositions, the laminin polymerization in an acidic medium in the presence of a divalent cation, preferably Ca²⁺, and the subsequent injection into the damaged area to provide the claimed therapeutic activity. The acidic polymer thus formed would have regenerative and anti-inflammatory capacity and would be able to restore neuronal plasticity lost during development to cerebral cortex explants of born animals, in addition to promoting the morphological regeneration of nervous tissue, as well as the functional recovery of spinal cord injury in mammalians. According to this document, laminin can be extracted from the murine EHS (Engelbreth-Holm-Swarm) sarcoma, or it can be recombinant human laminin, or laminin extracted from human placenta, or even a combination of these laminins. However, the document does not provide information regarding the methodology for extracting and purifying laminins. The laminin polymer formed under the conditions disclosed in this patent document was later called polylaminin by the inventors, and it is under clinical study in Brazil for the treatment of acute phase spinal cord injury (https://ensaiosclinicos.gov.br/rg/RBR-9dfvgpm).

Thus, there is a latent need for efficient methods for laminin extraction and purification in substantial quantities, applicable on industrial scale, whose methods allow obtaining the protein with considerable yield and purity, to enable the application of laminins in manufacturing of compositions for therapeutic use.

The first laminin extraction process identified in the state of the art is dated 1979, when Timpl et al. identified and named laminin as the main component of the tumor matrix of rat EHS sarcoma and, in normal tissues, located it in the basement membrane (Timpl, R., Rohde, H., Robey, P. G., Rennard, S. I., Foidart, J. M., & Martin, G. R. (1979). Laminin—a glycoprotein from basement membranes. The Journal of Biological Chemistry, 254 (19), 9933-9937). The authors described a process for obtaining laminin from this tumor which includes an extraction step, consisting of homogenization of the material in neutral buffer and NaCl (3.4 M NaCl, 0.05 M Tris-HCl, pH 7.4, at 4° C.), followed by centrifugation and further homogenization (10 to 20 volumes (w/v) of 0.5 M NaCl, 0.05 M Tris-HCl, pH 7.4). Next, a step of impurity precipitation, especially collagen, by the addition of 1.7 M NaCl. Finally, in the purification step, the supernatant was subjected to dialysis (2 M Urea; 0.05 M Tris-HCl, pH 8.6), anion exchange chromatography (DEAE-cellulose), concentration by ultrafiltration, molecular exclusion chromatography (Agarose A), further dialysis (0.05% acetic acid or 0.4 M NaCl, 0.05 M Tris-HCl, pH 7.4), concentration, and lyophilization.

Other processes in the state of the art for extracting laminin from EHS sarcoma have been identified, as in 1983, by Palm e Furcht (Palm, S. L., & Furcht, L. T. (1983). Production of laminin and fibronectin by Schwannoma cells: cell-protein interactions in vitro and protein localization in peripheral nerve in vivo. The Journal of Cell Biology, 96 (5), 1218-1226). Like the previous one, this process included an extraction step with homogenization (3.4 M NaCl, 0.01 M phosphate buffer, pH 7.4, 4° C.), centrifugation and further homogenization (0.5 M NaCl, 0.01 M phosphate, pH 7.4), followed by an impurity precipitation step with 1.7 M NaCl and laminin precipitation with 30% ammonium sulfate. The centrifugated precipitate was resuspended and dialyzed (0.5 M NaCl, 0.01 M phosphate, pH 7.4), subjected to molecular exclusion chromatography (Sephacryl S-300), dialysis (0.14 M NaCl, 0.01 M phosphate buffer, pH 7.4), affinity chromatography (Heparin-Sepharose), further dialysis (0.5 M NaCl, 0.01 M phosphate, pH 7.4) and, finally, filtration on a 0.45 μm filter and storage at −70° C. Although the methods described above are useful for providing laminin from rat EHS sarcoma, their efficiency could not necessarily be extrapolated to the laminin extraction from other tissues, such as human placenta, a material which is considered clinical waste and recognized as a source of laminins, especially those made up of subunits A, M, beta1, and beta2, such as isoforms 111, 211, and 221 (Rohde, H., Wick, G., & Timpl, R. (1979). Immunochemical characterization of the basement membrane glycoprotein laminin. European Journal of Biochemistry, 102 (1), 195-201; Foidart, J. M., Bere, E. W., Jr, Yaar, M., Rennard, S. I., Gullino, M., Martin, G. R., & Katz, S. I. (1980). Distribution and immunoelectron microscopic localization of laminin, a noncollagenous basement membrane glycoprotein. Laboratory Investigation, 42 (3), 336-342; Ohno, M., Martinez-Hernandez, A., Ohno, N., & Kefalides, N. A. (1983). Isolation of laminin from human placental basement membranes: amnion, chorion and chorionic microvessels. Biochemical and Biophysical Research Communications, 112 (3), 1091-1098). In this regard, according to Risteli and Timpl (1981), although the use of neutral buffer leads to the extraction of approximately 80% of intact laminin in the natural state from the EHS sarcoma matrix, the same effect may not be true for other tissues due to the presence of covalent and noncovalent interactions between basement membrane structures (Risteli, L., & Timpl, R. (1981). Isolation and characterization of pepsin fragments of laminin from human placental and renal basement membranes. The Biochemical Journal, 193 (3), 749-755. https://doi.org/10.1042/bj1930749). Thus, aiming to extract laminin from human placenta, the authors used a pepsin digestion step to solubilize the P1 fragment of laminin. Next, the process included precipitation steps with NaCl (1.2 M to 2 M to 4 M), purification by anion exchange chromatography (DEAE-cellulose), dialysis, collagen removal by the action of collagenase, and molecular exclusion chromatography.

However, the method described by Risteli e Timpl (1981), has the drawback of not recovering laminin in its entirety, but rather fragments from pepsin digestion. On the other hand, from the perspective of identifying common features, it is observed that the methods described so far, both for extraction from EHS sarcoma and from placenta, converge toon the use of selective precipitation techniques with varying NaCl concentrations (salting in and salting out), molecular exclusion chromatography, and anion exchange chromatography.

To overcome the problem of recovering laminin in fragments from the human placenta, Ohno et al. (1983) developed a non-degradative method from the placental basement membrane of dissected tissues from the amnion, chorion, and chorionic microvessels, without the need of previous digestion by proteases. After preparing the three tissues with NaCl, EDTA, in the presence of the protease inhibitors phenylmethylsulfonyl fluoride (PMSF) and N-ethylmaleimide (NEM), laminins were extracted through sequential treatment with: 1) 0.5 M NaCl containing 5 mM PMSF, 1 mM NEM; 2) 8 M urea in 0.05 M phosphate buffer, pH 7.0; and 3) 8 M urea with 2% 2-mercaptoethanol, and 2% sodium dodecyl sulfate, with centrifugations and dialysis between each treatment. The authors managed to extract intact laminins from the three tissues, however in the presence of potentially toxic substances for pharmaceutical use, such as protease inhibitors and reducing agents. NEM, for example, can inconveniently and irreversibly change the native structure of proteins with reduced cysteines in solution.

Dixit (1985), in turn, developed an extraction process in which laminin can be obtained in its intact form from human placenta in the presence of 10 mM EDTA. (Dixit S. N. (1985). Isolation, purification and characterization of intact and pepsin-derived fragments of laminin from human placenta. Connective Tissue Research, 14 (1), 31-40. https://doi.org/10.3109/03008208509089841). In this process, the pre-washed placenta was homogenized in neutral buffer (0.02-M Tris-HCl pH 7.4), 1 M NaCl and 3% Triton X-100 in the presence of 10 mM EDTA, which solubilized the laminin. The supernatant was subjected to two cycles of salting out with 4 M NaCl. For purification, the resuspended and dialyzed precipitate was subjected to the steps of anion exchange chromatography (DEAE-cellulose), molecular exclusion chromatography (Agarose A-5M-Void), dialysis and concentration (PM10 Diaflo). Between 17 and 20 mg of purified laminin were recovered from 1 kg of wet placental tissue, which indicates, according to the study, that the placenta may be a good source of human laminin.

Paulsson et al., in 1987, also applied EDTA as a laminin extracting agent from rat EHS sarcoma, in which laminin was complexed to nidogen (another protein characteristic of the basement membrane), excluding the salting out step and maintaining the molecular exclusion chromatography (Bio-Gel A5m or Sepharose Cl-6B) (Paulsson, M., Aumailley, M., Deutzmann, R., Timp1, R., Beck, K., & Engel, J. (1987). Laminin-nidogen complex. Extraction with chelating agents and structural characterization. European Journal of Biochemistry, 166 (1), 11-19).

Back to the human placenta, the patent document WO1991011462A2, entitled “Merosin, nucleic acids encoding, fragments and uses thereof” describes a process for merosin, a laminin isoform, extraction from human placenta, which includes a protein solubilization step in neutral buffer with 10 mM EDTA, and precipitation with 4 M NaCl, or a saturating concentration of 40% ammonium sulphate. The precipitate was subjected to molecular exclusion chromatography (Sepharose 6B) and anion exchange chromatography (DEAE cellulose) steps, in which merosin was eluted with 0.2 M NaCl.

U.S. Pat. No. 5,019,087, entitled “Nerve regeneration conduit”, relates to tubular devices containing human placental laminin useful in the in vivo regeneration of nervous tissue or damaged nerve, and described a process for extracting said laminin. The process is carried out at 4° C. and EDTA is used in low concentrations as protease inhibitor, together with NEM. Urea, a chaotropic agent, is used for extraction immediately after washing the placenta with NaCl and Tris, to remove blood elements. Next, the centrifuge supernatant is dialyzed, and the extract is concentrated using affinity chromatography (heparin) and precipitation with a saturating concentration of 30% ammonium sulphate.

In 2000, Champliaud et al described a method for obtaining laminins from placenta, which comprises extraction in the presence of the protease inhibitors PMSF and NEM, precipitation with a saturating concentration of 30% ammonium sulphate and purification that included anion exchange chromatography (DEAE cellulose), molecular exclusion chromatography (Gelatin-Sepharose), and immunoaffinity chromatography (pAb G4-G5 Sepharose→laminin alpha-1; MAb 545 Sepharose→laminin beta-1) steps (Champliaud, M. F., Virtanen, I., Tiger, C. F., Korhonen, M., Burgeson, R., & Gullberg, D. (2000). Posttranslational modifications and beta/gamma chain associations of human laminin alpha1 and laminin alpha5 chains: purification of laminin-3 from placenta. Experimental Cell Research, 259 (2), 326-335). It is important to highlight that the use of immunoaffinity chromatography, despite being widely present in small-scale laminin purification process in the literature, is not the most appropriate from an industrial production point of view. Furthermore, the presence of protease inhibitors in the medium is undesirable in processes to obtain products for pharmaceutical applications, due to their toxicity.

Gorelik et al (2001), in turn, carried out the laminin extraction from placenta with neutral buffer, EDTA and NaCl, precipitation with 4 M NaCl and, purification p only using molecular exclusion chromatography (Sephacryl S-300). Although they do not report the mass of laminin obtained from the extract, the authors highlighted that the results of the SDS-PAGE analysis, colored with silver, revealed some other bands, in addition to the bands referring to the laminin and nidogen fractions that have appeared in the gel colored with Coomassie blue (Gorelik, J. V., Cherepanova, O. A., Voronkina, I. V., Diakonov, I. A., Blinova, M. I., & Pinaev, G. P. (2001). Laminin-2/4 from human placenta is a better adhesion agent for primary keratinocytes than laminin-1 from EHS sarcoma. Cell Biology International, 25 (5), 395-402).

Hacketal et al (2018) developed a methodology for isolating laminin isoform 111 that comprises an extraction step with Tris-NaCl buffer combined with non-denaturing precipitation of the protein with 30% ammonium sulfate and quick steps of tangential flow filtration (Hackethal, J., Schuh, C. M. A. P., Hofer, A., Meixner, B., Hennerbichler, S., Redl, H., & Teuschl, A. H. (2018). Human Placenta Laminin-111 as a Multifunctional Protein for Tissue Engineering and Regenerative Medicine. Advances in Experimental Medicine and Biology, 1077, 3-17). According to this article, the average amount of laminin 111 recovered after isolation was 175±35 mg of laminin for every 100 g of wet basal tissue. One big drawback of this methodology is the need to remove the chorion, a serosa attached to the placental tissue, whose removal is difficult and laborious, being an obstacle to scaling up and applying it on an industrial scale.

The analysis of the state of the art clearly shows the tendency to use some main procedures for extraction and purification of laminin from placenta. In addition to selective precipitation, especially salting out with saturating concentration of ammonium sulfate (~30-40%) or sodium chloride (~3.4-4 M) or salting in with sodium chloride for removal especially of collagen (1.7 M), chromatographic techniques are widely used for purification steps. For example, molecular exclusion chromatography stands out due to the distinct size of laminin, but if applied without association with another chromatographic technique it can result in an unsatisfactory degree of purity. Affinity chromatographies, especially immunoaffinity, are also among the preferred ones in the state of the art, however, they involve the use of huge amount of antibodies, which can make the process more laborious, as well as more expensive. Furthermore, chromatographic resins functionalized with chemical groups instead of antibodies are easier to maintain, which makes the process more convenient to carry out on an industrial scale. Ion exchange chromatography is also an usual technique among laminin purification processes and, in a convergent manner, the choice of anion exchange chromatography over cation exchange one seems to be a consensus for laminin purification.

Apparently contrary to this tendency, patent document WO 1997/47652, entitled “Purification of soluble laminin 5”, discloses a laminin purification process including a cation exchange chromatography step. However, actually, the described protocol clearly follows the teachings of the state of the art, since the anion exchange chromatography technique is also used, as would be expected, in conventional laminin purification process.

However, it was observed by the present inventors that laminin extracted from human placenta, if purified by an anion exchange chromatography process, did not preserve its acid polymerization capacity and, therefore, resulted in a product unsuitable for therapeutic use in the treatment of injuries of the central nervous system, mainly spinal cord injuries, along the lines of the present invention. Therefore, there is a need for new methods to obtain laminin with preserved acid polymerization capacity.

In this regard, the present invention aims to provide an extraction process and a purification process of laminin that overcome the need in the state of the art for methods with good yields and industrial application and that, mostly, result in a purified product, that is laminin with preserved acid polymerization property, since this characteristic is essential to enable its therapeutic application, including in pharmaceutical compositions and kit for the treatment of central nervous system injuries, mainly spinal cord injuries.

### SUMMARY OF THE INVENTION

The present invention relates to an extraction process and a purification process of laminin from human placenta, said processes intends to obtain purified laminins with good yield and purity, to enable their use in the manufacture of therapeutic use compositions, and whose acid polymerization capacity to generate polylaminin is preserved. The present invention also relates to an acid polymerization process of purified laminins obtained through the process disclosed in this invention and to the polylaminin thus obtained. In addition, the present invention relates to the use of purified laminins obtained by the purification process in the preparation of pharmaceutical compositions, as well as a kit that allows the extemporaneous preparation of polylaminin for the therapy of central nervous system injuries, mainly spinal cord injuries.

The first embodiment of the present invention relates to a process for extracting laminin from human placenta to obtain a laminin-rich protein extract, wherein said process comprises steps of processing the placenta to obtain clean placental tissue without the need for removal of the chorion, homogenization of the placental tissue, extraction of laminins with an extraction buffer, optionally filtration and precipitation of laminins by salting out to recover the laminin-rich protein extract.

The second embodiment of the present invention refers to a process for purifying laminins from a protein extract which is rich in laminins, in which said process comprises the steps of solubilizing the extract in buffer with a chaotropic agent, cation exchange and chromatography molecular exclusion chromatography to obtain purified laminins. In addition, this embodiment of the invention includes non-essential steps for the final processing, which comprises one or more selected steps of concentration, filtration, and fractionation.

The third embodiment of the present invention refers to a process for purified laminins polymerization, in which said process is carried out from purified laminins obtained by the purification process disclosed in this invention, or from purified laminins obtained by combining the processes of extraction and purification processes disclosed in this invention, or even from the purified laminins obtained by the combination of suitable extraction processes in the state of the art and the purification process disclosed herein, which have preserved their acid polymerization capacity and therapeutic application.

The fourth embodiment of the present invention refers to the use of purified laminins obtained by the purification process disclosed in this invention, or obtained by combining the extraction and purification processes disclosed in this invention, or, even obtained by combining any suitable extraction process of the state of the art and the purification process disclosed herein, in the preparation of pharmaceutical compositions, which are useful for the treatment of central nervous system injuries, mainly spinal injuries.

The fifth embodiment of the present invention relates to polylaminin obtained by acid polymerization of purified laminins produced from the purification process disclosed herein, or from the combination of the extraction and purification processes disclosed herein, or, even, from the combination of any suitable laminin extraction process from the state of the art and purification process disclosed herein.

The sixth embodiment of the present invention relates to kits comprising a first vial containing a preparation comprising purified laminins obtained by the purification process disclosed in this invention, a second vial containing an acidic preparation, and, additionally, a preparation of divalent cations in the first, second or in a third vial, and further instructions for the extemporaneous preparation of a polylaminin composition for the treatment of central nervous system injuries, particularly for the treatment of spinal cord injuries.

It has been surprisingly demonstrated by the present inventors that the extraction process described herein is simpler and less laborious than those described in the state of the art, and results in a protein extract enriched in intact laminins and free of potentially toxic substances, whose presence is contraindicated in products for therapeutic use. Furthermore, and even more surprisingly and unexpectedly, the inventors developed a purification process that uses cation exchange chromatography and identified that this technique, even in the absence of anion exchange chromatography step, was enough to provide purified laminin, contradicting to the teachings of the state of the art. Even more important, the inventors detected that the use of anion exchange chromatography, usual in the state of the art for the purification of placental laminins, changed the polymerization pattern of the purified laminins and resulted in a protein product unsuitable for therapeutic applications. On the other hand, the process of the present invention, which employs cation exchange chromatography as the only chromatographic method of ionic interaction, instead of anion exchange, results in a product with considerable yield and purity and preserved acid polymerization capacity.

The aspects and embodiments of the present invention will become clearer from the following descriptions.

### BRIEF DESCRIPTION OF THE FIGURES

FIG. 1 shows the Western Blot of the samples of evaluation of laminin precipitation by ammonium sulfate under conditions of 208, 25% and 30% saturating salt concentration. In the image, the indications “γ1” and “α2 C-term (80 kDa)” refer to the regular chains in laminins of isoforms 211 and 221. Note, from left to right, 1: molecular weight marker; 2: standard laminin; 3: 30% precipitate; 30% 5:4: supernatant; 25% precipitate; 6: 25% supernatant; 7: 20% precipitate; 8: 20% supernatant. The presence of laminins in the supernatant fractions resulting from precipitation at 25% and 20%, respectively, is observed in 6 and 8.

FIG. 2 is a representative diagram of the comparison of the extract mass (▪) and the percentage of laminin in the extract (Δ) as a function of the saturating concentration of ammonium sulfate (%) used in the precipitation step. The mass obtained (▪) increases linearly as the amount of salt used increases. The percentage of laminins in the extract (Δ) decreases from approximately 13% to 3% due to the increase from 30% to 40% of ammonium sulfate.

FIG. 3 is the representation of the 6% SDS-PAGE analysis of the main intermediate fractions of the laminin purification process in a typical batch. Note, from left to right, 1: Molecular weight marker (mwm); 2: standard laminin (standard); 3: laminin-rich protein extract after resuspension, as described in Example 4.1 (resuspended); 4: resuspended material after filtration, carried out as described in Example 4.2 (filtered resuspended); 5: partially purified material obtained in the CEX step, carried out as described in Example 4.3 (CEX eluate); 6: purified laminins; 7: purified laminins after final processing. An increase in the purity of the material can be noted throughout the purification process, evidenced by the isolation of bands referring to laminins, noticeable in comparison with standard laminin.

FIG. 4 is the complete cation exchange chromatogram (CEX) referring to the production of a typical batch, in which the injection steps, column washing at 7.0-7.1 mS/cm, elution at 14.0-14.1 mS/cm, removal of remaining impurities at 48.0-48.1 mS/cm and cleaning with 1 M NaOH (CIP) are indicated. In the Figure, the solid line represents the absorbance at 280 nm (A₂₈₀) and the dotted line represents the measured conductivity.

FIG. 5 is a section of the cation exchange chromatogram (CEX) referring to the production of a typical batch, in which the laminin elution region is highlighted. In the Figure, the solid line represents the absorbance at 280 nm (A₂₈₀) and the dotted line represents the measured conductivity. The arrow points out the increase in absorbance (A₂₈₀), indicating the beginning of fraction collection. The highlighted region indicates the fraction collected and used in the next step of purification.

FIG. 6 is the complete molecular exclusion chromatogram (SEC) on Cytiva HiLoad 16/600 Superose 6 pg column for typical batch production. The demarcated regions indicate the first (F1) fraction and the second (F2) fraction collected.

FIG. 7 are the molecular exclusion chromatograms (SEC) on Cytiva Superose 6 Increase 10/300 gl column of the comparative analysis of the elution profiles of the first fraction (F1—solid line) and second fraction (F2—dotted line). FIG. 7A is related to batch production in which it is observed that the first (F1) and second (F2) fractions eluted at similar retention times and, thus, both were included in the laminin pool. FIG. 7B relates to the batch production in which it is observed that the second fraction eluted at a retention time different from that of the first fraction, which indicates a high content of impurities, and resulted in the discard of the second fraction (F2).

FIG. 8 is the chromatogram resulting from the loop SEC molecular exclusion chromatography method, on a Cytiva HiLoad 16/600 Superose 6 μg column, with five consecutive injections of 5 mL. The chromatogram relates to the production of a typical batch, that is, whose purification process was satisfactory. The highlighted areas under the curve indicate the laminin-rich fractions collected to compose the laminin pool.

FIG. 9 relates to the anion exchange step (AEX) whose application was evaluated in the purification process of this invention. FIG. 9A is the chromatogram resulting from the AEX step. The arrow indicates the elution peak of laminins. FIG. 9B represents the result of the 6% SDS-PAGE analysis, in which it is possible to see the reduction of impurities in the sample after the anion exchange step. Note, from left to right, 1: Molecular weight marker (mwm); 2: Standard Laminin (standard); 3: Material eluted from the CEX step (material injected in the AEX step) (injected); 4: Flow through (injection collection and washing at 14 mS/cm) containing impurities removed; 5: Material eluted from the AEX step at 28 mS/cm, containing laminins (eluted).

FIG. 10 represents the analysis of laminins polymerization obtained in purifications with and without the AEX step of the same protein extract. FIG. 10A shows the particle size distribution of the samples at neutral pH using the technique and DLS (Dynamic Light Scattering), and it is possible to observe that both samples have a similar average size and distribution. FIG. 10B shows the particle size distribution in DLS of samples at acidic pH, and it is possible to observe that the particle increasing which is typical of polymerization is observed only for laminins obtained in purification without AEX step. Laminins subjected to the AEX step, in turn, showed a partial polymerization profile at acidic pH. FIG. 10C shows Pulldown analysis via 6% SDS-PAGE. In this analysis, “C” are centrifuged (large centrifugable polymers) and “S” are supernatants (trimeric laminins or small non-centrifugable oligomers). It is noted that the proportion of centrifugable polymers at acidic pH is reduced with the introduction of the AEX step.

### DETAILED DESCRIPTION OF THE INVENTION

The present invention relates to an extraction process and a purification process of laminin from human placenta, said processes intended to obtain laminin with significant yield and purity, and with preserved acid polymerization capacity to generate polylaminin, as well as referring to the use of purified laminins thus obtained in the manufacture of pharmaceutical compositions for the therapy of central nervous system injuries, mainly spinal cord injuries, to an acid polymerization process of purified laminins of the invention, as well as polylaminin obtained by acid polymerization and a kit whose components comprise purified laminins, obtained according to the invention, an acidic preparation, divalent cations and instructions for extemporaneous preparation.

“Laminin” or “laminins” are synonymous and refer to the family of laminin isoforms. The term refers to a particular laminin isoform only when specified in this description. In a preferred but non-limiting embodiment, laminins are mainly isoforms 221 and 211, whose subunits are detected in placental tissue (Rohde, H., Wick, G., & Timpl, R. (1979). Immunochemical characterization of the basement membrane glycoprotein laminin. European Journal of Biochemistry, 102 (1), 195-201; Foidart, J. M., Bere, E. W., Jr, Yaar, M., Rennard, S. I., Gullino, M., Martin, G. R., & Katz, S. I. (1980). Distribution and immunoelectron microscopic localization of laminin, a noncollagenous basement membrane glycoprotein. Laboratory Investigation, 42 (3), 336-342; Ohno, M., Martinez-Hernandez, A., Ohno, N., & Kefalides, N. A. (1983). Isolation of laminin from human placental basement membranes: amnion, chorion and chorionic microvessels. Biochemical and Biophysical Research Communications, 112 (3), 1091-1098).

The term “polymerization” is defined, within the scope of this invention, as the process of laminin aggregation, especially in vitro, and encompasses said aggregation process under different conditions, including as described in the state of the art, whether by reaching laminin critical concentration in solution, by laminin adhesion to lipid surfaces, by contact of laminins with acidic medium in the presence or absence of divalent cations or, even, according to other in vitro laminin aggregation methodologies known in the state of the art.

The term “acid polymerization” is defined, within the scope of this invention, as the laminin polymerization exclusively when subjected to an acidic medium. In particular, it is characterized as the process of laminin aggregation when mixed with an acidic preparation, or when directly diluted in an acidic preparation, preferably in vitro, which leads to the immediate formation of high molecular weight laminin aggregates. According to the invention, the pH of said process must be acidic, preferably the pH is between 4.0 and 5.5, more preferably the pH is between 4.2 and 4.4. Even more preferably, the acidic polymerization occurs in the presence of a divalent cation, which in a preferred embodiment of the invention is Ca²⁺.

The terms “laminin polymer” or “polymer” are defined, within the scope of this invention, as an aggregate of subunits, wherein said subunits are laminins, linked through non-covalent bonds. Thus, despite not being a polymer from a chemical point of view, the terms are used in this document to define the aforementioned aggregates in accordance with their previous use in the scientific literature (Yurchenco, P. D., & Cheng, Y. S. (1993). Self-assembly and calcium-binding sites in laminin. A three-arm interaction model. The Journal of Biological Chemistry, 268 (23), 17286-17299; Barroso, M. M., Freire, E., Limaverde, G. S., Rocha, G. M., Batista, E. J., Weissmüller, G., Andrade, L. R., & Coelho-Sampaio, T. (2008). Artificial laminin polymers assembled in acidic pH mimic basement membrane organization. The Journal of Biological Chemistry, 283 (17), 11714-11720). These terms, in the context of obtaining them in an acidic medium, are applied to define polylaminin or corresponding to polylaminin within the scope of the invention.

“Polylaminin” is defined, within the scope of this invention, as a nano- and micro-structured network formed in vitro by non-covalent interactions between laminins, with central nervous system regeneration and anti-inflammatory activity. In particular, it is defined as a laminin polymer obtained by acid polymerization. In general, polylaminin obtained in vitro mimics the laminin organization in the cell membrane in vivo, although they are not identical given the absence of interaction with membrane receptors (Barroso, M. M., Freire, E., Limaverde, G. S., Rocha, G. M., Batista, E. J., Weissmüller, G., Andrade, L. R., & Coelho-Sampaio, T. (2008). Artificial laminin polymers assembled in acidic pH mimic basement membrane organization. The Journal of Biological Chemistry, 283 (17), 11714-11720).

Still according to the present invention, the term “protein extract” or “laminin-rich protein extract” refers to the extract obtained from the extraction process described in this invention and which is a precipitated material, generally wet and of pasty appearance, suitable for application in subsequent purification steps, also included in the scope of this invention. Despite of the fact that, in this description, the terms are applied specifically to the laminin extract obtained from the extraction process disclosed in the invention, the technician can alternatively apply selective precipitation and extraction processes known in the state of the art to obtain the laminin-rich protein extract, not yet fully purified, which will be subjected to the purification process revealed herein. In particular, the protein extracts obtained by state of the art methodologies which are considered suitable for application in the purification processes described here are those which are rich in intact laminins and free from substances considered unacceptable for application in pharmaceutical compositions.

A “physiologically acceptable liquid”, according to the scope of the present invention, is defined as a solution that does not present toxicity at the concentrations used, and is suitable for washing, resuspension, dissolution and/or homogenization of cells, tissues, proteins and biological materials, and which is preferably ultrapure water, saline solution (NaCl 0.9% w/v) or a physiologically acceptable buffer selected from the group comprising phosphate, citrate, acetate, histidine, tris and PBS (Phosphate-Buffered Saline) buffers.

A first embodiment of the present invention relates to a process for extracting laminin from human placenta to obtain a laminin-rich protein extract, wherein said process comprises the steps of:
(i) processing the placenta to obtain clean placental tissue;
(ii) placental tissue homogenization and separation of the homogenized tissue from the resulting fluids;
(iii) laminin extraction from placental tissue;
(iv) optional filtration; and
(v) selective precipitation of laminins in solution resulted from step (iii) or (iv) by salting out to obtain the laminin-rich protein extract.

The extraction process disclosed in this invention is efficient in providing a protein extract from human placenta that is enriched in intact laminins and which is faster and simpler to perform than the processes of the state of the art. Furthermore, the process disclosed herein provides an extract suitable for application in subsequent purification steps, which results in laminins suitable for therapeutic application, especially for use in pharmaceutical compositions for the treatment of central nervous system injuries, particularly for the treatment of spinal cord injury. Next, the technical and inventive aspects relating to the extraction process will become clear.

Placenta can be obtained, for example, by donation from a parturient with her express prior authorization, regardless the type of birth is natural or surgical. After collected, placenta can be stored and transported refrigerated or frozen form to the processing site. Placentas can be kept frozen, preferably at −20° C., between the time of collection and use. Placentas can be subjected to the extraction process either fresh, refrigerated or frozen, or even they can be subjected to more than one of these conditions. The process of obtaining placenta from parturient, however, is not part of the present claimed extraction process.

If frozen placentas are used, the material must be thawed prior to homogenization, which can be slow, under cooling, or quick. Preferably, the placenta is thawed quickly, at room temperature.

Step (i), processing the placenta, includes procedures to remove unwanted elements and wash the placental tissue. Processing is carried out in a contamination-free environment, preferably in laminar flow. The step is preferably carried out under cooling, at temperature between 2 and 8° C., preferably at 4° C.

According to the scope of the present invention, “unwanted elements” include outer membranes, traces of amnion and umbilical cord, but do not include the chorion. The chorion is a serosa whose removal is difficult and laborious, but it is reported in state-of-the-art extraction processes. The inventors have surprisingly identified that the extraction process whose placenta processing step does not require the removal of the chorion is easier and faster to perform, and results in laminin of a quality compatible with that desired and in satisfactory amount, without the need of additional or substitute extraction steps.

Placental tissue is washed with a physiologically acceptable liquid, preferably with saline solution (NaCl 0.9% W/V). Less ultrapure preferably, water is used as a physiologically acceptable liquid, since it may cause unwanted coagulation in the material. The washing liquid, preferably saline solution, is used at a cooled temperature, which is preferably between 2 and 8° C., most preferably 4° C. Before being subjected to washing, the placental tissue is cut into pieces whose shape and dimensions allow the permeation of physiologically acceptable liquid to remove blood, blood clots, and fluids released during the processing and cutting of said tissue. Preferably, the tissue is cut into cubes. In a preferred embodiment of this invention, the tissue is cut into cubes of approximately 5 cm and washed with saline solution. Washing is repeated twice, or until the tissue is free of blood and blood clots.

Step (ii), when the placental tissue is homogenized and the homogenized tissue is separated from the resulting fluids, includes homogenization of the tissue itself and its washing.

The homogenization procedure is carried out using the processed tissue obtained in step (i), to which a physiologically acceptable liquid is added at a cooled temperature, which is preferably between 2 and 8° C., most preferably 4° C. The liquid is preferably ultrapure water, in 2:1 ratio (200 ml of water for each 100 g of tissue), in which viscosity is optimized for homogenization, but a technician may be able to choose another proportion that allows a suitable homogenization without unwanted increase in material dilution. After adding the liquid, the suspended tissue is homogenized, preferably in a blender. Alternatively, homogenization can be carried out by other devices known in the state of the art which promote uniform fragmentation of the tissue. The procedure is carried out for no less than 1.5 minutes, but preferably, the procedure is carried out for no less than 4 minutes, which can be continuous or intermittent. In a preferred embodiment, the procedure is carried out with ultrapure water, in a blender for 4 minutes, divided into 2-minute cycles. The inventors of the present invention realized that numerous, long, and vigorous cycles using the blender are unnecessary, and the period between 1.5 and 4 minutes is enough for satisfactory fragmentation of the material and, mainly, without degradation of the laminins due to excessive homogenization. As an additional advantage of the present process, despite the suggestion of many processes in the state of the art, the inventors did not add toxic protease inhibitors substances during extraction, keeping the material free from potentially harmful additives for pharmaceutical use, such as, ethylmaleimide (NEM) and phenylmethylsulfonyl fluoride (PMSF). Despite the absence of these inhibitors, the developed process resulted in the recovery of intact laminins suitable for the applications included in the scope of the present invention.

The washing procedure is performed to remove fluids and blood released during the homogenization of the placental tissue suspension. First, the material is centrifuged to separate the homogenized solid tissue from the fluids contained in the supernatant, which must be discarded. The homogenized solid tissue that precipitates during centrifugation is resuspended in a cold physiologically acceptable liquid, preferably ultrapure water. According to the invention, the centrifugation/supernatant discard/resuspension cycle can be repeated to completely remove impurities in the supernatant and is followed by a final centrifugation step. In a preferred aspect of the invention, up to five centrifugation cycles are performed, including the final centrifugation. In an even more preferred aspect of the invention, three centrifugation cycles are performed, which include two centrifugation/supernatant discard/resuspension cycles and a final centrifugation. Centrifugation is preferably carried out at 3,000×g, for 5 minutes and at low temperature, preferably between 2 and 8° C., most preferably 4° C. During the development of the process of the present invention, the inventors found out that successive washing and centrifugation cycles provide the removal of fluids from the solid tissue, however, with progressive loss of laminins to the supernatant from the third centrifugation onwards. Thus, the inventors found out that three centrifugation cycles provide an adequate balance between the removal of said fluids and minimal loss of laminins to the supernatant.

Step (iii), extracting laminins from placental tissue, is applied to the homogenized solid tissue obtained in step (ii), and comprises the resuspension of said tissue in extraction buffer, followed by stirring and centrifugation to obtain the laminin-rich supernatant.

According to the scope of the present invention, the “extraction buffer” comprises Tris, NaCl, and EDTA. NaCl contributes to the laminin solubilization through the salting in phenomenon, while EDTA contributes to said solubilization by acting as a Ca²⁺ chelator, sequestering this ion, which plays an essential role in the formation and maintenance of laminin polymers in the basement membrane, from the medium, thus releasing laminins into the solution. In particular, the preferred buffer used in the invention comprises 50 mM Tris, 1.0 M NaCl, and 10 mM EDTA. The buffer pH is preferably between 7.3 and 8.1, more preferably the pH is 7.4. The extraction occurs in buffer in low temperature, preferably between 2 and 8° C., most preferably 4° C.

For resuspension, the mass of placental tissue and the volume of buffer are preferably in a 1:2 ratio (w/v). Extraction is carried out by stirring the homogenized tissue suspension in extraction buffer, for a period between 8 and 16 hours, at a temperature between 2 and 8° C. Agitation is preferably slow. The term “slow agitation” refers to agitation without vortex formation in the suspension. During the development of this new process, the inventors discovered that vigorous agitation, that is, under conditions in which vortex is formed, has the disadvantage of leading to the degradation of laminins and, therefore, must be avoided.

Recovery of supernatant that is enriched in soluble laminins is performed by centrifugation. In a preferred embodiment of the process of the present invention, the suspension is centrifuged at 15,000×g during 30 minutes at low temperatures, preferably the temperature is between 2 and 8° C., most preferably at 4° C.

Step (iv), filtration of the supernatant that is enriched in laminins obtained in step (iii), is optional and can be carried out to remove the particulate material remaining in the laminin solution, as noticed by the high turbidity of the solution. The inventors realized, in the course of this development, that the extraction process is possible to be carried out without filtration step (iv) but provides a material with remaining solid residues. Thus, to improve the quality of the laminin solution that will be subjected to the precipitation step (v), the inclusion of the filtration step (iv) was evaluated. In a preferred embodiment of the invention, filtration is carried out with depth filters, even more preferably, filtration is carried out with depth filters whose nominal filtration degree is between 0.05 and 2 μm.

The final step of the extraction process, that is, step (v), is the selective precipitation of laminins, which is applied to the laminin solution resulting from step (iii) or step (iv) and, the laminin-rich protein extract is recovered. This step is carried out through salting out, in which the addition of salt promotes aggregation and consequent protein precipitation due to the selective reduction of solubility.

Laminin precipitation is carried out with ammonium sulfate salt. Alternatively, another salt can be used whose application in salting out is known in the state of the art. Ammonium sulfate can be added in the form of crystals or as saturated solution.

The saturating concentration of ammonium sulfate used in step (v) is between 20 and 80% ammonium sulfate, preferably, the saturating concentration is between 20 and 40% ammonium sulfate, more preferably, the saturating concentration is 30%, in which the inventors found out that there was maximum precipitation of laminins and reduced precipitation of the other proteins of the extract.

Precipitation is carried out by adding salt slowly to the solution under slow stirring, ensuring homogenization without salt accumulation at the bottom of the container. The temperature in this step is low, preferably in between 2 and 8° C., most preferably 4° C. Precipitation under these conditions is maintained for a period in which an increase in turbidity is visibly noted, which indicates laminin precipitation. In particular, precipitation can be maintained for an excessive period of time, 2 hours for example, in order to guarantee maximum precipitation of said laminins.

After the precipitation period, when the solution turbidity is increased due to the presence of precipitated proteins in suspension, mainly laminins, the suspension is centrifuged. In a preferred embodiment of the invention, the suspension is centrifuged at 15,000×g during 30 minutes under low temperature, preferably between 2 and 8° C., most preferably 4° C. The supernatant is discarded and the precipitate, called laminin-rich protein extract, within the scope of the present invention, a pasty material containing the precipitated proteins, is recovered. It was found that this extract i is suitable for subsequent steps in the purification process, which can be carried out immediately or, alternatively, the extract can be frozen until required for use, preferably at −20° C.

The laminin-rich protein extract can be characterized regarding to the presence of laminins and the laminin isoforms that compose it. In particular, the characterization can be carried out using analytical techniques based on the specificity of antigen-antibody binding. Preferably, techniques employing polyclonal anti-laminin antibodies or monoclonal antibodies, whose binding is specific to a particular α, β or γ chain can be used. More preferably, characterization can be done by Western blot. Alternatively, other analytical methods available in the state of the art can be used. Additionally, the extract can be characterized with regard to the relative increase in the amount of laminins to the detriment of other proteins originally present in the original material, qualifying said extract as laminin-rich or, enriched in laminins, or, equivalently within the scope of the invention, laminin-rich. Preferably, techniques for protein detection and/or quantification such as A₂₈₀, monitoring of the protein profile in two- or three-dimensional gels, such as SDS-PAGE or even analytical molecular exclusion chromatography can be used. Alternatively, other analytical methods available in the state of the art, and whose application is known by the technician can be used. In any case, within the scope of the present invention, detection can essentially be carried out to confirm that laminins are the main components of the protein extract when compared to the starting material, characterizing it as an enriched extract.

A second embodiment of the present invention refers to a process for purifying laminins from a laminin-rich protein extract, wherein said process comprises the steps of:
(a) solubilization of laminin-rich protein extract in a resuspension buffer in the presence of a chaotropic agent;
(b) cation exchange chromatography; and
(c) molecular exclusion chromatography to obtain purified laminins.

Additionally, purified laminins obtained in step (c), in the context of pharmaceutical applications and within the scope of the present invention, can be subjected to final processing steps. These procedures are not intended to actually purify laminins from impurities of protein nature, and they are included as efforts to increase the final concentration of purified laminins, to reduce the volume of purified laminins and/or to remove impurities, mainly non-protein impurities, such as reducing the microbiological load.

Within the scope of the present invention, the term “purified laminins” refers to a solution comprising laminins isolated through the process disclosed herein and said process comprising maximizing the removal of other proteins from the material, considered protein impurities, as well as possible non-protein impurities, while maintaining the maximum possible quantity of laminins in the final material.

The purification process disclosed in this invention is, surprisingly and contrary to the teachings of the state of the art, applicable for the purification of substantial amounts of laminins from human placenta, with considerable yield and purity. The unexpectedly, inventors, observed that the purification of laminins through this new process, which does not include an anion exchange chromatography step usual in the state of the art, leads to obtention of adequately purified laminins. Even more unexpectedly, the inventors identified that the techniques used in the present process result in purified laminins with preserved acid polymerization capacity, differently to the laminins obtained when usual state of the art methods were used, more precisely, when anion exchange chromatography technique was employed. Acid polymerization is an essential and fundamental characteristic for the therapeutic application of laminins in compositions and treatments for central nervous system injuries, particularly spinal cord injuries within the scope of the present invention. Next, the technical and inventive aspects relating to the purification process of the present invention will become clearer.

Step (a), solubilization of the laminin-rich protein extract in the presence of a chaotropic agent, is carried out to reduce the conductivity of the protein solution and to impair interactions between laminin and protein impurities in solution.

The laminin-rich protein extract of this step is any extract obtained from human placenta by extraction methods, or by extraction and precipitation methods, including similar ones already known in the state of the art, provided that it is enriched in laminins. In a preferred embodiment of the invention, the laminin-rich protein extract is obtained through the extraction process disclosed within the scope of this invention. In case of using alternative laminins enriched protein extracts, i.e., obtained by other methods described or usual in the state of the art, step (a), solubilization in a chaotropic agent, can be omitted or adapted to the conditions for obtaining the alternative extract, according to the obvious knowledge of the technician about this matter. For example, if the extract is obtained diluted or lyophilized, in non-pasty form, with high or low ionic strength, with the presence or absence of a chaotropic agent, for example, as a result of the extraction steps of the state of the art, the technician, as a result of his/her intrinsic knowledge, can adapt centrifugation, dissolution and chaotropic agent addition procedures to convert said alternative extract into an extract suitable for application in the subsequent purification step (b), without any prejudice to the scope claimed in the present invention.

Before and after each purification step, preferably, the total protein concentration is evaluated, estimated, for example, by absorbance of the sample at 280 nm (A₂₈₀), or, less preferably, by other methods of the state of the art such as colorimetric methods, like the Bradford method. Furthermore, preferably, the protein profile is evaluated by SDS-PAGE, and the presence of laminins is detected by immunological detection techniques, such as Western blot. Chromatographic methods can also be used to monitor the purification steps, such as Size Exclusion Chromatography (SEC), which can be combined to dynamic light scattering analytical techniques, such as SEC-MALS (MALS=Multiple Angle Light Scattering). Alternatively, the technician can monitor the process by applying other techniques available in the state of the art.

In this step (a) a “resuspension buffer” is used, which is defined as a physiologically acceptable buffer comprising a chaotropic agent. The physiologically acceptable buffer is preferably Tris buffer. The chaotropic agent is preferably urea. The dissolution of the laminin-rich protein extract in the resuspension buffer promotes the reduction of the solution conductivity salinity and favors the interaction between laminins and cation exchange resin in the following chromatographic step. In addition, the chaotropic agent acts to destabilize protein structures and impair the interaction between laminins and protein impurities, which would be undesirable in the next step. In a preferred embodiment of the invention, the resuspension buffer is 20 mM Tris, containing 2 M urea and preferably pH of 7.5.

Resuspension buffer is added to the laminin-rich protein extract and the mixture is slowly stirred. Preferably, the mixture is stirred using a magnetic stirrer. Within the scope of the present invention, in a preferred embodiment, the resuspension buffer is added until the conductivity reaches a value lower than 7 mS/cm, under stirring, which is maintained until maximum solubilization of the precipitate. Preferably, stirring is maintained for 15 minutes.

Step (b)—cation exchange chromatography—is carried out for the purification of a laminin-rich solution such as the solution obtained in step (a), which comprises impurities of protein nature, and aims to increase the purity of the material by separating laminin from other proteins still present in solution, thus increasing the quality of the material injected in the final purification. This separation is carried out based on the different interactions between the charges of different proteins and the charges of the cation exchange resin, which depends on the variety of conditions of ionic strength used in the process, which is mediated by the concentration of positive ions in solution.

Most proteins, including laminins, have an overall negative charge when at nearly neutral pH. This would favor, at first glance, the interaction of laminins with anion exchange resins instead of cation exchange resins, under these pH conditions. In fact, the state of the art teaches the use of anion exchange chromatography almost unanimously when there is option for ion exchange chromatography in the purification of placental laminins. However, contrary to the teachings of the state of the art, the inventors found that laminins not only interact with the cation exchange resin, but also that an anion exchange step is not necessary for their satisfactory purification. In addition, but as relevant as, inventors observed that, using the anion exchange chromatography technique to purify placental laminins, as taught by the state of the art, despite obtaining a successful purification step, the laminins recovered had their acid polymerization capacity reduced or lost. Thus, laminins were not suitable for use in pharmaceutical compositions for treatment of spinal cord injuries, which is another aspect of the present invention. On the other hand, the inventors unexpectedly found out that, by omitting the anion exchange chromatographic step and maintaining the cation exchange chromatography step in the present process, not only purified laminins were obtained, but also with preserved acid polymerization capacity. Next, cation exchange chromatography procedure of step (b) is detailed according to the scope of the present invention.

Prior to subjecting the laminin as obtained in step (a) to the cation exchange chromatography of step (b), the material resulting from step (a) can be prepared. The preparation, which is known by the technician, can be, for example, a filtration step. Thus, within the scope of the present invention, filtration is performed on the resuspended protein extract obtained in step (a), or an equivalent material as per the previous description, with the purpose of removing protein precipitates, cellular debris and residual tissue which may still remain in said extract. Such removal is necessary to ensure the quality of the following steps and contributes to the longevity of the chromatographic resins. The preparation, therefore, is not essential for increasing the material purification degree in relation to laminins and is not considered fundamental to the purification process itself.

Filtration is carried out on filters with 0.5 μm or less. Preferably, filtration is carried out on 0.1 μm filters. Even more preferably, filtration is carried out using a combination of pre-filter and filter. In a preferred embodiment of the invention, filtration is carried out using a 0.5 μm pre-filter and a 0.1 μm filter. Filtration can be carried out with the aid of a peristaltic pump. After the filtration step, the solution must be clear and free of particles.

Thus, within the scope of the present invention, the material obtained in step (a), or equivalent, properly prepared as is known by the technician, preferably filtered, is subjected to the cation exchange chromatography of step (b). Preferably, a cation exchange resin suitable for separating large biomolecules, such as laminin, is used, according to the resin manufacturer's specifications. For example, the resin base matrix is highly porous with a large pore size, which reduces steric hindrance and favors the adsorption of large molecules. In an embodiment of the invention, a resin composed of a hydrophilic porous polymer or copolymer is used. In a preferred embodiment, a cross-linked copolymer of allyl dextran and N, N-methylene bisacrylamide, or a cross-linked agarose matrix or a membrane of stabilized reinforced cellulose is used. Regarding to functionalization, the matrix is linked to negatively charged groups, preferably, the matrix is functionalized with sulfopropyl (SP) or methyl sulfonate(s), which are strong cation exchangers. In a preferred embodiment of the invention, the resin is a cross-linked copolymer of allyl dextran and N, N-methylene bisacrylamide functionalized with —SO₃, more preferably functionalized with sulfopropyl.

In the aforementioned chromatographic step, two buffers are used. The resuspension buffer, as defined in this description, and the “elution buffer”, which differs from the resuspension buffer as it has greater conductivity, which is needed to the elution of proteins bound to the chromatographic resin. Preferably, the higher conductivity is obtained by increasing the ionic strength in the elution buffer and is due to the presence of NaCl. Preferably, the elution buffer comprises Tris buffer, urea and NaCl. In a preferred embodiment of the invention, the elution buffer is 20 mM Tris, 2 M urea, 1 M NaCl and the pH is 7.5.

The cation exchange chromatographic step is carried out according to the technician's knowledge. The material obtained in step (a), or equivalent, properly prepared as is known by the technician, preferably filtered, is introduced into the chromatographic column in resuspension buffer and elution is carried out by gradually increasing the eluent conductivity, which is achieved by adding elution buffer in scheduled amounts. The eluted fractions are collected depending on the increase in the total protein concentration, which can be evaluated by A₂₈₀/and those whose laminin concentration is high in relation to the total protein concentration are used in subsequent steps. Monitoring the concentration of laminin in the aforementioned fractions, to select fractions suitable for proceed the purification process, is carried out using techniques available in the state of the art, preferably is carried out by analytical molecular exclusion chromatography of a sample.

In a preferred embodiment of the invention, the chromatographic column is equilibrated with a buffer at conductivity between 7.0 and 7.1 mS/cm, in which impurities of low interaction with the resin are eluted. Next, laminin elution is carried out at conductivity between 14.0 and 14.1 mS/cm. Finally, the elution of high interaction impurities and washing of the column are carried out at conductivity between 48.0 and 48.1 mS/cm. Given this non-limiting embodiment, a technician can adapt the chromatographic conditions depending on the chosen resin.

The eluted fraction with partially purified laminins can be stored under refrigerated conditions, preferably at temperature between 2 and 8° C., until being used in the subsequent purification step.

Additionally, but not necessarily, the eluted fraction containing partially purified laminins, obtained in step (b) of cation exchange chromatography, can be concentrated to reduce its volume and facilitate the following step. For example, when the A₂₈₀ absorbance of the eluted fraction containing partially purified laminins is less than 1.00 AU (absorbance units), the said eluted fraction can be concentrated.

Step (c), molecular exclusion chromatography, is carried out from the material eluted in the cation exchange chromatography step (b), that is, from the eluted fraction containing partially purified laminins. Its purpose is to separate laminin from impurities remaining in the solution based on apparent size, using inert porous resins.

Preferably, a molecular exclusion resin suitable for separating large biomolecules, such as laminin, is used, according to the resin manufacturer's specifications. Preferably, the resin has pores with a nominal separation range of 5 to 5,000 kDa. In an embodiment of the invention, the resin is a hydrophilic porous polymer or copolymer. In a preferred embodiment of the invention, the resin is a highly cross-linked agarose matrix.

In molecular exclusion chromatography, only a buffer is used as the mobile phase, whose flow rate in the column is constant. Preferably, the buffer used is PBS.

Step (c)—molecular exclusion chromatography—is carried out according to the technician's knowledge. The eluted fraction containing partially purified laminins obtained in step (b) is inserted into the column already packed with balanced molecular exclusion resin. The buffer is then injected at a constant flow rate, and the eluted fractions are collected and tested for the presence of laminins and total proteins. Monitoring estimated concentrations of f total proteins and laminins is carried out as explained in this description. Those fractions whose laminin concentration is increased in relation to the total protein concentration when compared to previous steps of the process are the fractions of interest.

Fractions collected as a result of step (c) comprise the purified laminins.

The final processing of purified laminins is an additional purification process, since it does not have the main objective of increasing the purity of the purified laminins with respect to the protein nature. Final processing is used to assign a higher concentration to the purified laminins and/or to remove any remaining impurities, especially those of a non-protein nature of the material, for example, to reduce the microbiological load. The following description will make clear the purposes and execution of the final processing procedures, in which the technician chooses, depending on the need observed during the process execution, to apply the final processing, as well as to practice one or more of the aforementioned processing procedures.

Final processing of purified laminins may include a concentration procedure. Said procedure is carried out with the purpose of correcting the concentration of the laminin solution to a target concentration, where this target concentration is higher than that obtained as a result of step (c) of the purification process. Within the scope of the present invention, as part of the process of using laminins as an active ingredient in pharmaceutical compositions, the fractions eluted in the chromatography of step (c) are preferably subjected to concentration. To achieve this, concentrators are used and the A₂₈₀ measurement is corrected to a value greater than 0.25 AU. The concentration method to be used is in accordance with the description of the state of the art, which includes, for example, the use of concentrators or tangential flow filtration (TFF). Preferably, concentrators with a cutoff of 30 kDa or 50 kDa are used. In a preferred embodiment of the present invention, concentrators with a polyethersulfone (PES) membrane are used. At the end of the concentration procedure, the purified and concentrated laminin solution is recovered.

Final processing of purified laminins may include a filtration procedure. This procedure is carried out with the aim of reducing the microbiological load of the material. The filtration procedure is carried out in a contamination-free medium, preferably in a laminar flow, and sterilizing filters are used, preferably filters of 0.2 μm. In a preferred embodiment of the process of the present invention, filtration is carried out with sterile polyethersulfone membrane filters.

Final processing of purified laminins may include a fractionation procedure. In said procedure, the purified, optionally concentrated, optionally filtered laminins are fractionated in sterile containers, preferably polypropylene tubes.

After the purification procedure, the purified laminins recovered in step (c) or, alternatively, the purified laminins subjected to final processing are analyzed with regard to the total protein concentration, e.g. using A₂₈₀, and with regard to the final laminin concentration, e.g. using analytical SEC and SEC-MALS. The final yield of the purification process is also calculated. The material recovered in step (c), or, alternatively, in step (d), can then be stored frozen until use. Preferably, the material is stored at −80° C.

As observed by the present inventors, the purified laminins obtained through the purification process disclosed herein are suitable for therapeutic application in the treatment of nervous system injuries, especially for the treatment of spinal cord injuries, since they have preserved their acid polymerization capacity. In view of this property, the present invention is presented in other embodiments, disclosed below, in which, for simplification purposes, the term “purified laminins” is used in reference to laminins obtained through the purification process defined by the steps (a) to (c), subjected or not to final processing.

A third embodiment of the present invention refers to a process for purified laminin polymerization, in which said process is carried out from purified laminins obtained by the purification process disclosed in this invention, or from purified laminins obtained by combining the extraction and purification processes disclosed in this invention or, even from the purified laminins obtained by combining any suitable laminin extraction process in the state of the art and the purification process disclosed herein.

Said polymerization process is carried out by contacting purified laminins with an acidic preparation, whose pH of the resulting composition is, preferably, between 4.0 and 5.5, more preferably the pH is between 4.2 and 4.4. In an embodiment of the present polymerization process, purified laminins are mixed with an acetic acidic preparation. In a preferred embodiment, purified laminins are added to a 30 mM acetic acidic preparation. The acidic preparation may also contain an osmolality adjusting agent used in enough amount to achieve a physiological osmolality range suitable for local application in spinal cord injuries, preferably the osmolality range is between 270 and 330 mOsmol/kg. The preferred osmolality adjusting agent is sodium chloride. In a preferred embodiment, the acidic preparation comprises 144.87 mM sodium chloride. The inventors of the present invention have surprisingly observed that purified laminins obtained through the process claimed in the present invention are suitable for the acid polymerization process, unlike laminins obtained by purification processes with an anion exchange chromatography step, which are usual in the state of the art.

An additional feature of the polymerization process claimed is the presence of cations in the polymerization medium. Preferably, the process is carried out in the presence of divalent cations. In a more preferred embodiment of the present invention, the divalent cation is Ca²⁺. Furthermore, in an embodiment of this invention, calcium ions are contained in the acidic preparation. In a preferred embodiment, the acidic preparation comprises acetic acid and calcium chloride, the latter preferably at a concentration of 1.2 mM. In a more preferred embodiment of the invention, the acidic preparation comprises 30 mM acetic acid, 1.2 mM calcium chloride and 144.87 mM sodium chloride.

A fourth embodiment of the present invention relates to the use of purified laminins in the preparation of polylaminin pharmaceutical compositions, wherein the purified laminins are obtained from the purification process disclosed in this invention or from the combination of the extraction and purification process in disclosed this invention or, furthermore, from the combination of any suitable laminin extraction process in the state of the art and the purification process disclosed herein. As a result of the laminin preserved acid polymerization property, as obtained by the improved purification process described herein, the use of said purified laminins in pharmaceutical polylaminin compositions is made feasible. As a consequence, the application of aforementioned pharmaceutical compositions for the treatment of central nervous system injuries, mainly spinal cord injuries, is feasible.

A fifth embodiment of the present invention refers to polylaminin obtained by acid polymerization of purified laminins, in which the purified laminins are obtained from the purification process disclosed in this invention, or from the combination of the extraction and purification processes disclosed in this invention, or even from the combination of any suitable laminin extraction process in the state of the art and the purification process disclosed herein. Said polylaminin is obtained by a polymerization process as described in a previous embodiment of the present invention, which comprises the contact of said purified laminins with an acidic medium, preferably in the presence of a divalent cation.

As demonstrated by the inventors, purified laminins obtained by other methods in the state of the art, more precisely laminins obtained using an anion exchange chromatography step, had their acid polymerization capacity reduced or eliminated, and they did not polymerize into polylaminin when in an acidic medium, which makes the polylaminin obtained by the extraction and purification processes described herein another object of the present invention.

A sixth embodiment of the present invention relates to kits essentially comprising a first vial containing a preparation comprising purified laminins and a second vial containing an acidic preparation. These kits are useful for the extemporaneous preparation of a polylaminin composition for the treatment of central nervous system injuries, particularly for the treatment of spinal cord injuries. In the context of the present invention, the acidic preparation may comprise an osmolality adjusting agent, preferably sodium chloride, used in sufficient quantity to a physiological osmolality range suitable for local application in spinal cord injuries. In addition, said kit comprises a preparation with a divalent cation, which may be contained in the first vial, in the second vial or even in an additional third vial of the kit.

Within the scope of the present invention, the term “extemporaneous preparation” refers to the preparation of a mixture between the components of the first vial, the second vial and, optionally, the third vial of the kit just before its therapeutic application. Preferably, the preparation is carried out in vitro. Thus, the kit may additionally comprise instructions for the extemporaneous preparation of a polylaminin composition for the treatment of central nervous system injuries, particularly for the treatment of spinal cord injuries.

The kits of the present invention, as well as polylaminin, have proven being useful in the recovery of patients with spinal cord injury in clinical studies, so the processes described here are of great importance for public health, especially in resolving a kind of injury which has no therapeutic options, until the present moment.

The superiority of the processes, use, kit, as well as polylaminin disclosed in the present invention will become clearer through the following examples. The examples represent a preferred form of carrying out the invention and, therefore, should not restrict the scope of the invention.

### EXAMPLES

### Example 1: Obtaining Laminin-Rich Protein Precipitate or Extract from Human Placenta

The laminin extraction process from human placenta, according to the present invention, was carried out under the following steps.

Human placentas were obtained from parturient, with their express prior consent, by anonymous, unpaid and voluntary donation. They were selected under strict eligibility criteria based on clinical history, hereditary diseases and infectious diseases. At the time of the childbirth, the donated placenta was placed in properly sealed primary and secondary plastic packaging and then stored at −20° C. to maintain the integrity of the material until processing.

Using laminar flow, the material was thawed in water at room temperature, the external membranes, remaining amnion and umbilical cord were removed and discarded, and then the placental tissue was cut into cubes and washed with refrigerated saline solution (NaCl 0.9% w/v) to eliminate clots and fluids. The chorion was not removed.

The clean placental tissue was homogenized in an industrial blender for four minutes after adding 200 mL of ice-cold ultrapure water for each 100 g of tissue. The fluid and blood released during homogenization were removed by centrifugation at 3,000×g for 5 minutes at 4° C., discarding the supernatant and repeating the centrifugation cycle twice with the solid material recovered.

For the laminins extraction, each 100 g of centrifuged solid tissue was resuspended in 200 mL of ice-cold extraction buffer (50 mM Tris, 1.0 M NaCl, 10 mM EDTA, pH 7.4). The suspension was kept under slow stirring, with magnetic stirrer, for a period between 8 and 16 hours, at a temperature between 2 and 8° C. Further, the liquid phase or supernatant containing soluble laminin was separated from the remaining tissues by centrifugation (15,000×g, 30 minutes, 4° C.).

Finally, laminins were precipitated from the supernatant recovered in the previous step using 30% saturating salt concentration at 4° C. Therefore, 16.98 g of ammonium sulfate for every 100 mL of solution were slowly added, under slow stirring, until complete solubilization and then the solution was kept stirring for two hours at a temperature between 2 and 8° C. After this period, high turbidity was observed due to the formation of protein precipitates in suspension. The laminin-rich protein precipitate or extract was recovered by centrifugation (15,000×g, 30 minutes, 4° C.) and stored at −20° C.

### Example 2: Determination of the Ideal Saturation Concentration of the Salt for Laminin Precipitation by Salting Out Using Ammonium Sulfate

The ideal condition for laminin precipitation with ammonium sulfate was determined through tests with saturation concentrations of the salt in the range from 20% to 80% at 4° C.

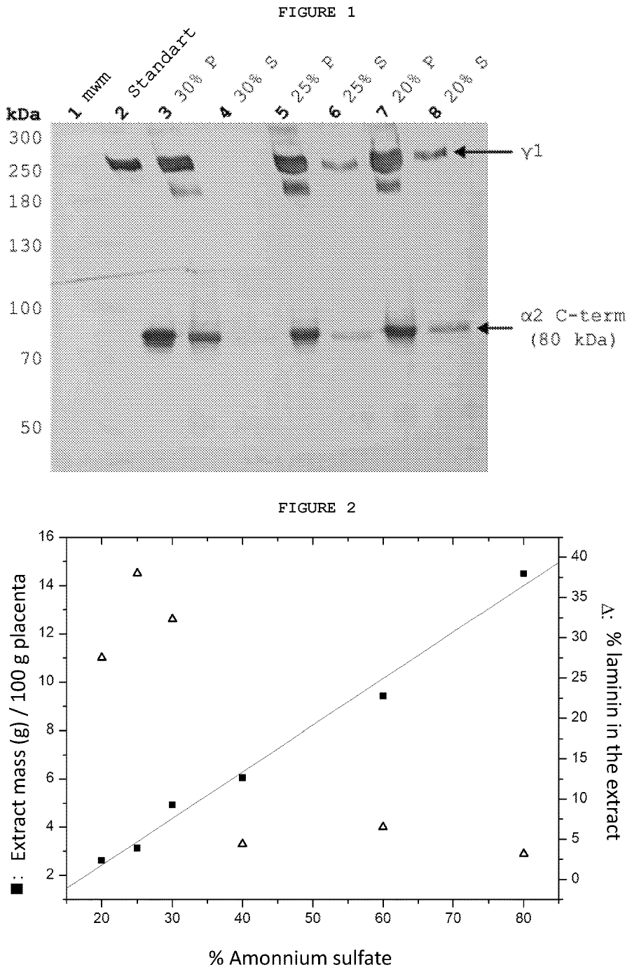

At concentrations of 20% and 258, the presence of laminins was detected in the supernatant fraction (FIG. 1), an evidence of incomplete precipitation of laminins from the solution, which was not observed at concentrations of 30%, 40%, 60% and 80%. The analysis of the precipitated fraction, in its turn, revealed an increase in the co-precipitation of impurities with laminin at concentrations starting at 40% (FIG. 2), which is undesirable as it reduces the quality of the laminin-rich protein extract that will be the starting material for subsequent steps in the purification process. From the results, the researchers concluded that 30% of the saturation concentration of ammonium sulfate is the ideal condition to be used in the extraction process, as there is a balance between maximum precipitation of laminins, which are not detected in the supernatant, and reduced co-precipitation of impurities, estimated by the total mass of the precipitate, which is advantageous in subsequent purification steps. However, this step of the process can be carried out under saturation conditions between 30% and 80%, since laminins are completely precipitated, even though they constitute a smaller percentage of the extract. Saturation conditions between 20 and 30% can also be used, even though the process yield is lower due to the loss of laminins in the supernatant fraction.

### Example 3: Optional Filtration Step Prior to Precipitation

The removal of solid residues by filtration prior to salt precipitation was evaluated in regard to the filters used. It was observed that depth filters are the most suitable among those evaluated to reduce the turbidity of the suspension and increase the quality of the final protein extract, according to results shown in Table 1.

TABLE 1
Filters tested in the purification step.

| | | Filtering Area | |
|---|---|---|---|
| Filter | Model | (m²) | Result |
| Filter with | Merck-Millipore | 0.017 (47 mm) | Immediate |
| 0.1 μm | OptiScale Express | | clogging (less |
| PES membrane | SHR 0.5/0.1 μm | | than 2 mL |
| with 0.5 μm | | | filtered) |
| pre-filter | | | |
| Capsule with | Merck-Millipore | 0.048 (XL 300) | Reduced |
| 0.1 μm | SHR OptiCap | | turbidity, |
| PES membrane | Capsule XL | | clogging with |
| with 0.5 μm | 0.5/0.1 μm | | less than |
| pre-filter | | | 100 mL |
| | | 0.29 (XL 5) | Reduced |
| | | | turbidity, |
| | | | clogging with |
| | | | around 700 mL |
| Depth Filter | 3M ZetaPlus 30S | 0.017 (47 mm) | Reduced |
| | | | turbidity, |
| | | | no clogging |
| | 3M ZetaPlus 60S | 0.017 (47 mm) | Reduced |
| | | | turbidity, |
| | | | no clogging |
| | 3M ZetaPlus 90S | 0.017 (47 mm) | Reduced |
| | | | turbidity, |
| | | | no clogging |

### Example 4: Purification of Laminin-Rich Protein Extract in Order to Obtain Purified Laminins

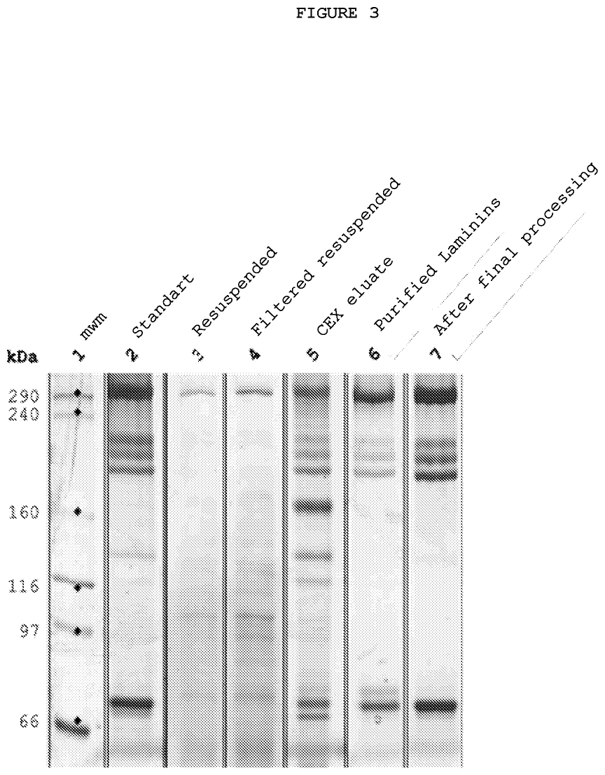

The laminin-rich protein extract obtained as described in the previous examples was subjected to the purification process. The increase in purity of laminins in the material through the following purification steps was evidenced by SDS-PAGE, as shown in FIG. 3. Other analytical methods performed are mentioned in the description of each step.

### Example 4.1: Resuspension

Up to three protein extracts coming from different placentas were used together in the same purification process. The flasks containing the protein extract were thawed at room temperature and diluted in Resuspension Buffer (20 mM Tris, 2 M Urea, pH 7.5) as described below.

The Resuspension Buffer was added to the flask containing the extract, which was gently shaken to release any extract adhered to the flask walls and then the flask was washed with buffer. The procedure was repeated with all flasks with extract, whose material was collected in a single container of appropriate size, which was subjected to stirring with a magnetic stirrer. Under slow stirring, the conductivity was monitored with a conductivity meter and the Resuspension Buffer was slowly added to the solution until the measured conductivity had reached the condition of <7 mS/cm. The solution was kept under slow stirring for 15 minutes at room temperature to ensure maximum solubilization of the extract. After solubilization, a sample was removed to evaluate total protein concentration by absorbance at 280 nm (A₂₈₀) and SDS-PAGE profile (FIG. 3).

### Example 4.2: Filtration

The suspension obtained according to the procedure described in EXAMPLE 4.1 was filtered to remove protein precipitates, cellular debris and residual tissue present in solution, as a preparation for subsequent chromatographic steps.

The suspension was pumped via a peristaltic pump into a filter capsule Merck-Millipore SHR OptiCap Capsule XL 3 0.5/0.1 μm, which has a 0.5 μm pre-filter, a 0.1 μm polyethersulfone (PES) filter and filtering area of 0.16 m². In case of clog during the process, the capsule was emptied, and a new capsule was used to complete the material filtration. After filtration, a clear, particle-free solution was obtained. A sample was taken to evaluate total protein concentration (A₂₈₀), molecular exclusion profile (SEC) and SDS-PAGE profile (FIG. 3).

### Example 4.3: Cation Exchange Liquid Chromatography (CEX)

Cation exchange chromatography uses resins functionalized with negatively charged groups that interact with positively charged solutes.

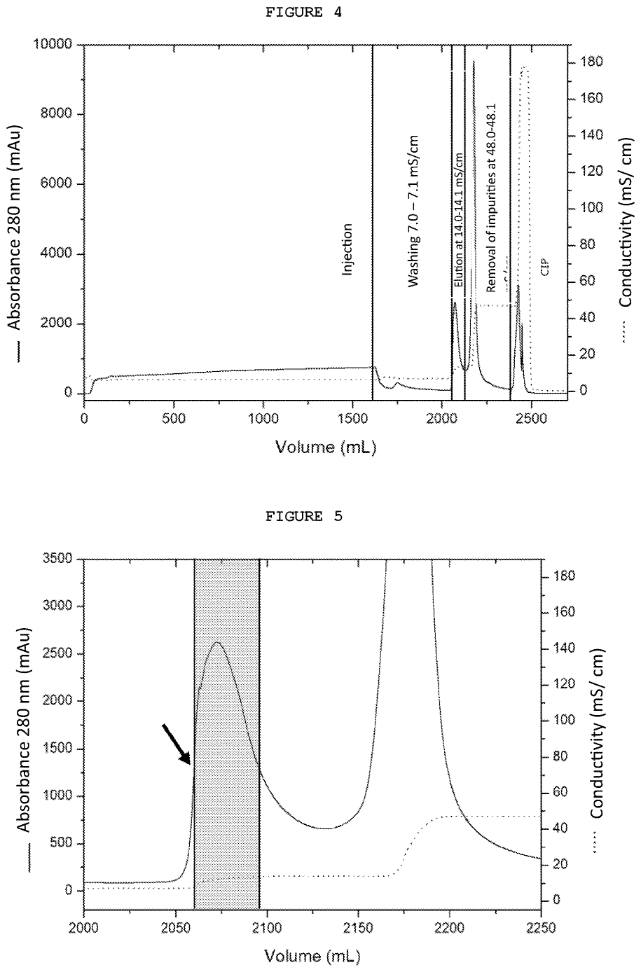

In this embodiment of the invention, purification was carried out with a Millipore Vantage L Laboratory Column VL 32×250 column, packed with 41 mL of Cytiva MacroCap SP resin. The column was installed in a Cytiva Äkta Pure chromatograph. During the process, mixtures of buffers A and B were used. Buffer A is the Resuspension Buffer and consists of 20 mM Tris and 2 M Urea, at pH 7.5. Buffer B is the Elution Buffer and consists of 20 mM Tris, 2 M Urea and 1 M NaCl, at pH 7.5. The proportions used in the mixtures of buffers A and B in each chromatographic step are described in Table 2, below, and the complete chromatographic process, carried out in accordance with this embodiment of the invention, is presented in FIG. 4. The conductivity ranges were determined by the inventors in a method they programmed into the chromatograph, which mixed buffers A and B, with a gradual increase in the salt concentration.

TABLE 2
Buffer solutions used in the CEX chromatographic
process depending on the mixtures to achieve the
appropriate target conductivity (σ) at each step.

| | | Approximate |
|---|---|---|
| | Target σ | percentage (%) |
| Step | (mS/cm) | of B |
| Column balance and elution | 7.0-7.1 | 5.4 |
| of impurities with low | | |
| interaction with the resin | | |
| Laminin elution | 14.0-14.1 | 12.9 |
| Elution of impurities with | 48.0-48.1 | 54.0 |
| high interaction with the | | |
| resin and column washing | | |

After packing, the column was balanced with a mixture of buffers A and B in the range of 7.0-7.1 mS/cm. After balance, an automated method was used to perform the material injection steps into the column, washing and laminin elution.

Using the chromatograph sample pump, the resuspended and filtered extract was injected into the column at a flow rate of 90 cm/h (12 mL/min). Injection continued until the air sensor associated with the sample pump had detected complete injection of the contents into the column, and then the sample pump and tubing were automatically emptied with the injection of resuspension buffer. Next, the column was washed with 10 column volumes (CV) of the mixture of buffers A and B with conductivity of 7.0-7.1 mS/cm, at a flow rate of 90 cm/h to elute impurities that have low interaction with the resin. The elution was monitored by absorbance at 280 nm, and the stable baseline indicated the elution of the aforementioned impurities, shown in FIG. 5.

Subsequently, laminins were eluted from the resin. The ratio between buffers A and B was changed to achieve conductivity of 14.0 to 14.1 mS/cm. An increase in absorbance at 280 nm, monitored by the chromatograph, indicates the beginning of protein elution, as can be seen in FIG. 5.

The collected fraction (FIG. 5), containing the initial 0.85 CV (35 mL) of the elution peak, encompassed the main fraction of laminins with a reduced amount of protein impurities. This fraction, therefore, went to the next purification step. Laminins were still detected in the remaining of the elution peak, but at a lower concentration and, in addition, a considerable amount of associated protein impurities was detected, so that it was considered inappropriate for further purification. Thus, the collected fraction was characterized in terms of total protein concentration (A₂₈₀) and SDS-PAGE profile (FIG. 3) and was then stored in a refrigerator at a temperature of 2-8° C.

Finally, after elution of the laminin-rich fractions of interest, the impurities remaining in the column were removed by changing the ratio of buffers A and B to achieve conductivity of 48.0-48.1 mS/cm. Next, the column was cleaned with 1 M sodium hydroxide and water and stored in 20% ethanol.

### Example 4.4: Concentration

In the concentration step, the material eluted from cation exchange was concentrated with 2 to 6 Merck-Millipore Amicon Ultra-15 concentrators, with a cutoff of 30 kDa or 50 kDa. The target volume was calculated using the equation:

$${V}_{target} = ( {C}_{eluted} * {V}_{eluted} ) / 1 , 35$$

Where V_{target}=target volume at the end of the concentration step (mL), C_{eluted}=concentration of total protein of the eluted material in the CEX step in A₂₈₀ (UA) and V_{eluted}=volume of the eluted material in the CEX step (mL).

The concentrators were filled, balanced and centrifuged at 3000×g at 4° C. for 10 minutes. After 10 minutes, the solutions in the concentrators were stirred with a pipette (to avoid aggregation at the bottom of the concentrator) and had their volume measured. The cycle was repeated until the target volume was achieved. The concentrated solution was then combined and had its concentration measured again by A₂₈₀. The concentrated eluted material was stored in a refrigerator at 2-8° C.

During the development of the invention, the inventors observed that the concentration step needed to be used when the purification process started from the protein extract of just one placenta, but it was unnecessary after scaling up, with extracts from three placentas, for example. It was observed that when the total protein concentration analyzed by A₂₈₀ reached a value equal to or greater than 1.35 AU, the material concentration process was too slow and resulted in protein aggregation in the sample. Scaling up, however, the material eluted from the CEX step had the total protein concentration analyzed by A₂₈₀ and values above 1.35 AU were observed, so the concentration step became unnecessary.

### Example 4.5: Molecular Exclusion Liquid Chromatography (SEC)

In this embodiment of the invention, purification was carried out with Cytiva Superose 6 prep grade resin, which has a nominal separation range of 5-5000 kDa and is suitable for the separation of high molecular weight molecules, such as laminins. The column used was HiLoad 16/600, 60 cm high and total volume of 120 mL of resin.

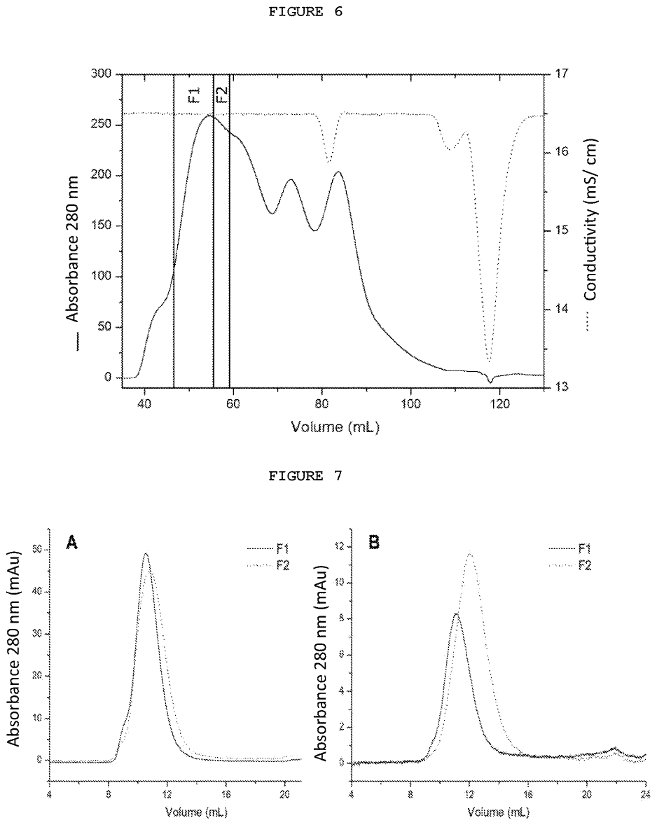

After packing, the column was balanced in PBS (Phosphate-Buffered Saline) and then 5 mL (4.17% CV) of material eluted in the CEX step (concentrated or not) were injected into the column at a constant flow of 1 mL/min (29.8 cm/h). After sample injection, the flow was maintained at 1 mL/min using PBS as the mobile phase of the chromatography. Fraction collection began 46.5 mL (0.3875 CV) after the beginning of the injection. The first 9.00 mL collected fraction contains the beginning and the main fraction of the laminin peak. Immediately afterwards, a second fraction of 3.46 mL was also collected. The complete chromatographic process, carried out according to this embodiment of the invention, is presented in FIG. 6.

The second fraction contains the final portion of the laminin peak and possible impurities of a protein nature, which may vary in ratio from batch to batch. To evaluate the quality of the material, the first and second fractions were analyzed via molecular exclusion chromatography (Cytiva Superose 6 Increase 10/300 gl column) and had their chromatographic profiles compared, as shown for two different batches in FIG. 7. When both fractions present similar elution profiles (FIG. 7A), that is, with coincident retention time and absence of secondary peaks, both fractions were used to compose the pool of purified laminins used in final processing, since there is evidence of few impurities in the second fraction. Otherwise (FIG. 7B), only the first fraction composed the pool and subsequent steps.

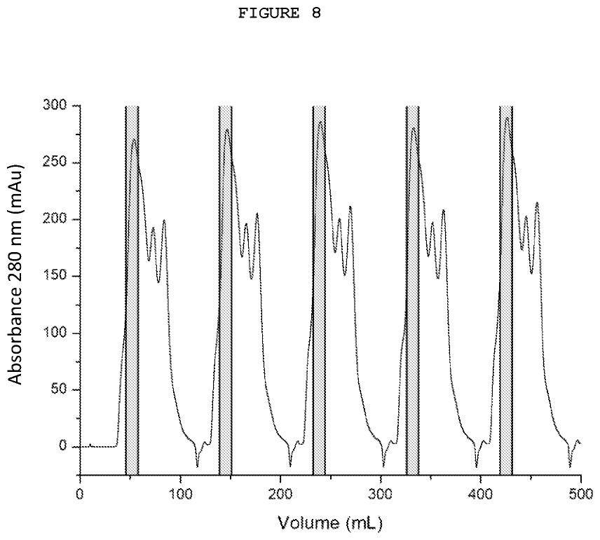

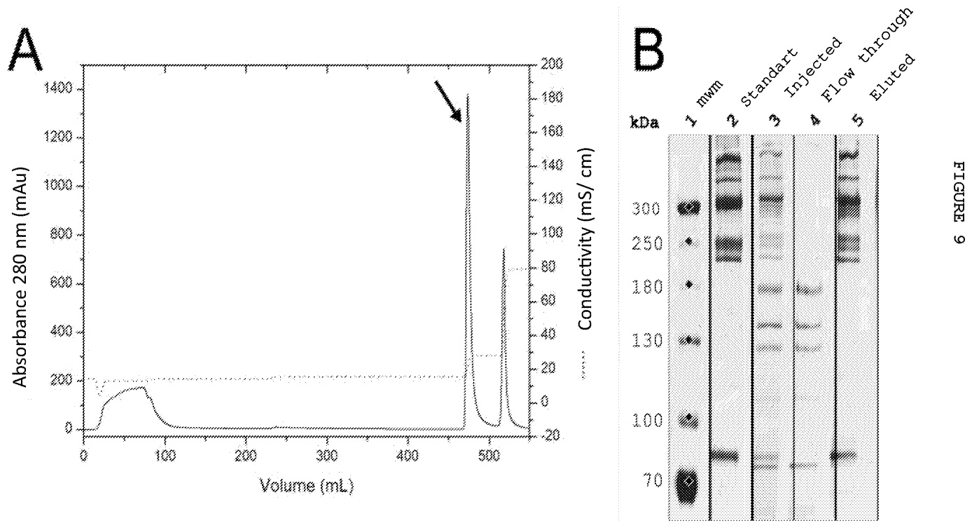

In this embodiment of the invention, the injected volume was limited to 5 mL, and multiple chromatographic runs were required to process all the material from the previous purification step. In general, for the cation exchange eluate that was not subjected to the concentration step, seven runs were required for full processing. To speed up the process, the inventors developed a chromatographic method in loop (FIG. 8), which performs material injections at pre-calculated times in order to compress multiple runs into just one. Using this method, the waiting time to complete each run and the residence time of the dead volume (time between injection and the beginning of elution of the first solutes) were eliminated. A technician can adapt sample injection to the most convenient column, resin and chromatographic methods based on knowledge of the state of the art.

Finally, the fractions selected for the laminin pool from multiple runs were pooled and characterized with regard to the theoretical concentration calculated from the A₂₈₀ of the chromatograms. The pool was stored in a refrigerator at 2-8° C. until the next step was carried out.

### Example 4.6: Concentration

After the molecular exclusion chromatography (SEC) step, the laminin pool is already in its final formulation (PBS), but it is still necessary to correct its concentration, in order to the A₂₈₀ value is greater than 0.25 UA. To achieve this, the target volume was calculated with the equation:

$${V}_{target} = ( {C}_{pool} * {V}_{pool} ) / 0 , 32$$

Where V_{target}=target volume at the end of the concentration step (mL), Cₚₒₒₗ=total protein concentration of the laminin pool eluted in the SEC step in A₂₈₀ (UA) and Vₚₒₒₗ=volume of the laminin pool eluted in the SEC step (mL).

The concentration procedure is similar to the procedure previously described. Two to six Merck-Millipore Amicon Ultra-15 concentrators were used, with a cutoff of 30 kDa or 50 kDa. The concentrators were filled, balanced and centrifuged at 3000×g, at 4° C. for 5 minutes. After 5 minutes, the solutions in the concentrators were shaken by pipetting and their volume measured. The cycle was repeated until the target volume was reached. Next, a measurement of A₂₈₀ was taken and the concentration was corrected to the range 0.30 to 0.34 AU, either with another centrifugation cycle or with dilution with PBS. The concentrated laminin pool was then stored in a refrigerator at 2-8° C.

### Example 4.7: Filtration, Fractionation and Storage

In a laminar flow, the concentrated laminin pool was filtered using sterile Millipore Millex GP Filter Unit 0.22 μm filters. Next, the filtered pool was fractionated in sterile polypropylene tubes and its final volume was recorded. One of the samples was used for final concentration analysis by A₂₈₀ and final yield calculation. The batch was frozen and stored in a freezer at −80° C.

### Example 5: Anion Exchange Liquid Chromatography (AEX)

This chromatographic step was tested for laminins as an intermediate purification step, with the aim of removing urea from the solution and increasing the material purity, thus increasing its quality for injection in the final purification step. The development was carried out in Cytiva MacroCap Q resin, through direct injection of the material obtained from CEX chromatography.

The AEX step was performed on a MacroCap Q 3 mL column. First of all, the material eluted from the CEX step was injected, followed by washing the column at a conductivity of 14 mS/cm to remove protein impurities that have low interaction with the resin and remove urea. The column was washed with buffer, whose conductivity is 20 mS/cm. Laminins, in its turn, were eluted with a buffer whose conductivity is 28 mS/cm and the corresponding fractions were collected. Finally, the remaining impurities that have high interaction with the resin were eluted with 1 M NaCl.

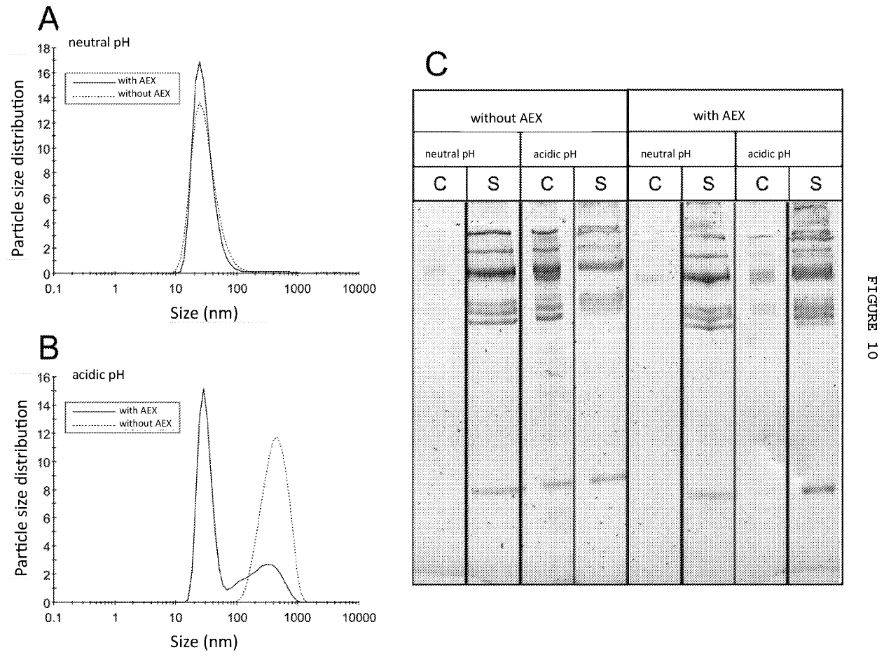

In terms of purification, the introduction of the AEX chromatographic step was successful (FIG. 9). For the test batch, the relative purity of the material increased from 25% to 63% after AEX and the recovery of 81% of the laminins from the sample was satisfactory. However, when analyzing the finished batch, it was observed that laminins decreased or lost their acid polymerization capacity, which makes their therapeutic use unfeasible. The loss of polymerization capacity was observed by two orthogonal techniques: DLS (Dynamic Light Scattering) and Pulldown, (FIG. 10). Another three batches produced introducing the AEX step showed the same reduction in acid polymerization capacity. For this reason, the use of the AEX step has been discarded.

1-51. (canceled)

52. A process for extracting laminin from human placenta to obtain a laminin-rich protein extract comprising:
(i) processing the placenta to remove unwanted elements, and washing to obtain clean placental tissue;
(ii) homogenizing the clean placental tissue and separating homogenized tissue from the resulting fluids;
(iii) extracting laminins from the homogenized placental tissue in extraction buffer consisting of 50 mM Tris, 1.0 M NaCl, 10 mM EDTA and having pH 7.4, under slow stirring, wherein the buffer volume and the placental tissue mass are in 2:1 ratio;
(iv) optional filtration; and
(v) selectively precipitating laminins in solution resulted from step (iii) or (iv) by salting out with ammonium sulfate salt at a saturating concentration of 30% to obtain a laminin-rich protein extract;
wherein step (i) does not require removal of the chorion from the placental tissue and wherein the process is carried out in the absence of protease inhibitors.

53. The process, according to claim 52, that is carried out using placenta frozen at −20° C. and further comprising thawing of the frozen placenta.

54. The process, according to claim 53, wherein the placenta is quickly thawed at room temperature.

55. The process according to claim 52, wherein the washing in step (i) is carried out with an ice-cold physiologically acceptable liquid.

56. The process according to claim 52, wherein homogenization in step (ii) is carried out in an industrial blender with a physiologically acceptable liquid.

57. The process according to claim 52, wherein in step (ii) the homogenized tissue is separated from the resulting fluids by centrifugation.

58. The process according to claim 52, wherein filtration in step (iv) is carried out through a depth filter.

59. A process for purifying laminins from a human placenta laminin-rich protein extract comprising:
(a) solubilizing human placenta laminin-rich protein extract in a resuspension buffer in the presence of a chaotropic agent;
(b) cation exchange chromatography which is employed as the only ion exchange chromatographic step in the process; and
(c) molecular exclusion chromatography to obtain purified laminins.

60. The process according to claim 59, wherein the human placenta laminin-rich protein extract is obtained by the process comprising:
(i) processing the placenta to remove unwanted elements, and washing to obtain clean placental tissue;
(ii) homogenizing the clean placental tissue and separating homogenized tissue from the resulting fluids;
(iii) extracting laminins from the homogenized placental tissue in extraction buffer consisting of 50 mM Tris, 1.0 M NaCl, 10 mM EDTA and having pH 7.4, under slow stirring, wherein the buffer volume and the placental tissue mass are in 2:1 ratio;
(iv) optional filtration; and
(v) selectively precipitating laminins in solution resulted from step (iii) or (iv) by salting out with ammonium sulfate salt at a saturating concentration of 30% to obtain a laminin-rich protein extract;
wherein step (i) does not require removal of the chorion from the placental tissue and wherein the process is carried out in the absence of protease inhibitors such as pheny-methylsulfonyl fluoride and N-ethyl-maleimide.

61. The process according to claim 59, wherein in step (a) the chaotropic agent is urea.

62. The process according to claim 59, wherein in step (a) the resuspension buffer is 20 mM tris, containing 2 M urea and pH 7.5.

63. The process according to claim 59, wherein cation exchange chromatography is carried out with a resin that is a cross-linked copolymer of allyl dextran and N,N-methylene bisacrylamide, or a cross-linked agarose matrix or a membrane of stabilized reinforced cellulose.

64. The process according to claim 59, wherein in step (b) cation exchange chromatography is carried out with resin functionalized with sulfopropyl (SP) or methylsulfonate(S) groups.

65. The process according to claim 59, wherein in step (b) elution of the laminin fraction is carried out with an ionic strength gradient.

66. The process according to claim 59, wherein in step (b) cation exchange chromatography is carried out with resin functionalized with sulfopropyl groups (SP), in which the material introduced into the column has a conductivity of 7 mS/cm and the elution of the laminin fraction occurs with an increase in the conductivity of the eluent to a range between 14.0 and 14.1 mS/cm by NaCl addition.

67. The process according to claim 59, wherein molecular exclusion chromatography resin has pores with a nominal separation range of 5 to 5,000 kDa.

68. The process according to claim 59, further comprising step d), wherein the laminins are concentrated using a concentrator with a cutoff of 30 kDa or 50 kDa.

69. A kit for the extemporaneous preparation of a polylaminin pharmaceutical composition for use in the treatment of spinal cord injury and central nervous system injuries, comprising a first vial that contains purified laminins obtained by the purification process as defined by claim 59, and a second vial that comprises an acidic preparation.

70. The kit according to claim 69, wherein the acidic preparation additionally comprises an osmolality adjusting agent.

71. The kit, according to claim 69, that further comprises a preparation of divalent cations.
